(19) United States
(12) Patent Application Publication
(10) Pub. No.: US 2016/0242680 A1
(43) Pub. Date: Aug. 25, 2016

(54) INTELLIGENT COMFORT LEVEL MONITORING SYSTEM

(71) Applicant: UMM AL-QURA UNIVERSITY, Makkah (SA)

(72) Inventors: Muhammad ARIF, Makkah (SA); Emad Felemban, Makkah (SA)

(73) Assignee: UMM AL-QURA UNIVERSITY, Makkah (SA)

(21) Appl. No.: 14/627,972

(22) Filed: Feb. 20, 2015

(51) Int. Cl. A61B 5/11; A61B 5/145; A61B 5/0205; A61B 5/00; A61B 5/024

(52) U.S. Cl. CPC A61B 5/1112; A61B 5/7271; A61B 5/02438; A61B 5/02055; A61B 5/14542

(57) ABSTRACT

A comfort level monitoring system monitors the physical and surrounding environment of an individual includes a wearable item to house a plurality of sensors, at least one physical pressure measuring sensor to detect an amount of external physical pressure exerted on a person wearing the wearable item, a first set of sensors to detect environmental data associated with an environmental comfort level of the person wearing the wearable item, a second set of sensors to detect oxygen ratios associated with the environment surrounding the person, a third set of sensors to detect total energy expenditure of the person, a user interface to receive personal physical data associated with the person, a fuzzy logic circuit to calculate, in response to received inputs, an overall comfort level associated with the person, and an activity alarm triggered when the overall comfort level falls below a predetermined threshold.

### BACKGROUND

The present disclosure relates generally to systems and methods for intelligently monitoring personal comfort level and recommending actions based on monitored activities.

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

As the population of the world is growing day by day, maintaining the public order in the crowded areas of the big cities is becoming very important. Some examples of crowded public areas include airports, railway stations, carnivals, concerts and sports events. Increasing numbers of people in small areas may create problems like physical injury and fatalities. Movement in the crowd affects the people differently depending on different age groups and different preexisting health conditions. Discomfort in the crowd may put stress on the people and ignite panicked behaviors. In extreme weather conditions, people may suffer extreme health conditions, including extreme discomfort and fainting.

What is needed are new crowd activity monitoring methods and systems that have advanced capabilities to monitor individual comfort levels in crowd to improve crowd management and security monitoring for large crowds.

### SUMMARY

A long standing and unfulfilled need for improved awareness of crowd and individual comfort levels as measures of public safety and enhanced crowd monitoring and management are addressed by the present disclosure. The present disclosure relates to a comfort level monitoring system that includes a wearable item to house a plurality of sensors, at least one physical pressure measuring sensor to detect an amount of external physical pressure exerted on a person wearing the wearable item, a first set of sensors to detect environmental data associated with an environmental comfort level of the person wearing the wearable item, a second set of sensors to detect oxygen ratios associated with the environment surrounding the person, a third set of sensors to detect total energy expenditure of the person, a user interface to receive personal physical data associated with the person, a fuzzy logic circuit to apply a fuzzy logic algorithm to calculate, in response to received inputs from the at least one physical pressure measuring sensor, the first set of sensors, the second set of sensors and the third set of sensors, an overall comfort level associated with the person, and an activity alarm to be triggered when the overall comfort level falls below a predetermined threshold.

The comfort level monitoring system also includes at least four physical pressure measuring sensors placed within the wearable item to maximize measurement of external physical pressure and wherein a different value weighting is assigned to each one of the at least four physical pressure measuring sensors depending on a location of the at least four physical pressure measuring sensor within the attire. Furthermore, in the comfort level monitoring system the first set of sensors includes at least one of a temperature sensor, a humidity level sensor, a wind speed sensor, and a solar radiation sensor and wherein the environmental comfort level is generated by non-linear aggregating circuitry configured to create an n dimensional space, for n input parameters.

In an exemplary embodiment, in the comfort level monitoring system the input parameters include wind speed information, solar radiation information, temperature information and humidity information when n=4, and the third set of sensors further includes at least one global positioning sensor (GPS) and at least one inertial navigation sensor (INS). The GPS and INS sensor data is used to predict an activity performed by the person and the total energy expenditure calculation is dependent on the predicted activity performed by the person. The predictive activity may further include walking, running, standing, and sitting.

In yet another exemplary embodiment, the comfort level monitoring system includes personal physical data associated with the person that includes body strength, age, body mass index (BMI) and gender; wherein a different value weighting is assigned to each one of the genders such that the generated overall comfort level is dependent on the weighting of the different genders. The activity abort alarm of the comfort level monitoring system may further instruct at least one individual to abort the physical activity currently in pursuit. The activity abort alarm further generates a crowd dispersion outline configured to ease specific congestion location within a venue.

In another aspect, the disclosure is directed to a computer-readable medium including instructions. The instructions, when executed by at least one programmable process, may cause the at least one programmable processor to perform operations. The operations may include detecting an amount of external physical pressure exerted on a person wearing the wearable item, detecting environmental data associated with an environmental comfort level of the person wearing the wearable item, detecting oxygen ratios associated with the environment surrounding the person, detecting total energy expenditure of the person, receiving personal physical data associated with the person, applying a fuzzy logic algorithm to calculate, generating an overall comfort level associated with the person, and triggering an alarm when the overall comfort level falls below a predetermined threshold.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a sample sensor configuration within a clothing attire according to an exemplary embodiment;

FIG. 2 is a sample sensor configuration within a clothing attire according to an exemplary embodiment;

FIG. 3 is a system overview of a data logger, transmission and pre-processing system and a given interaction configuration with different sensor blocks according to an exemplary embodiment;

FIG. 4 is a system overview of a comfort level expert system according to an exemplary embodiment;

FIG. 5 is a plot of vertical acceleration during walking mapped out over a time (t) in seconds according to an exemplary embodiment;

FIG. 6A illustrates sample environmental comfort level (ECL) calculations according to an exemplary embodiment;

FIG. 6B illustrate sample environmental comfort level (ECL) plotting according to an exemplary embodiment;

FIG. 7 is an environmental comfort level scale to determine comfort or discomfort levels according to an exemplary embodiment;

FIG. 8A is a sample activity logging and calculation algorithm according to an exemplary embodiment;

FIG. 8B illustrates an activity acceleration and GPS plot as a function of time according to an exemplary embodiment;

FIG. 8C illustrates a sample activity classification algorithm according to an exemplary embodiment;

FIG. 9A illustrates a comfort level expert system fuzzy word membership allocation according to an exemplary embodiment;

FIG. 9B illustrates a comfort level expert system according to an exemplary embodiment;

FIG. 9C illustrates a sample Fuzzification/defuzzification algorithm according to an exemplary embodiment;

FIG. 10 illustrates a comfort level expert system with activity abort recommendation according to an exemplary embodiment;

FIG. 11 illustrates a comfort level expert system with activity alarm generation according to an exemplary embodiment;

FIG. 12 illustrates an expert system management portal according to an exemplary embodiment; and

FIG. 13 illustrates a hardware diagram a device according to exemplary embodiments.

### DETAILED DESCRIPTION

Referring now to the drawings, wherein like reference numerals designate identical or corresponding parts throughout the several views.

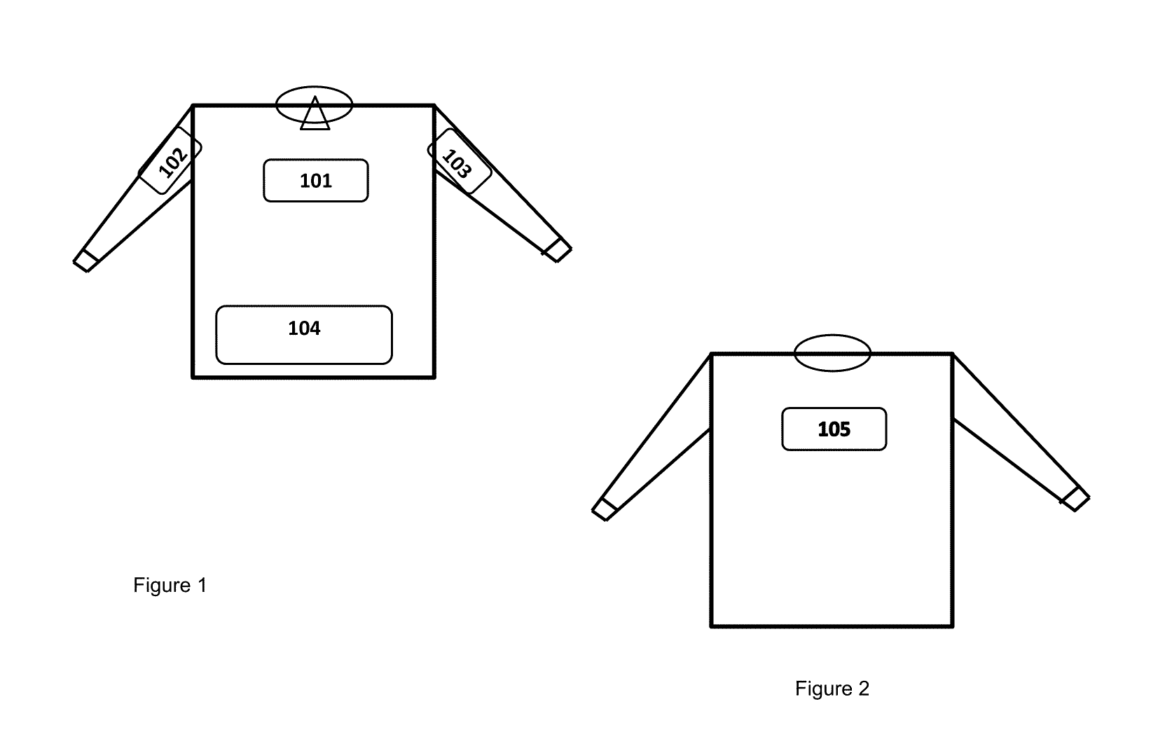

FIG. 1 is a sample sensor configuration within clothing item 100 according to an exemplary embodiment. The sensors 101, 102, and 103 are sensor circuits designed to generate a wide array of data regarding the environmental impact experienced by an individual wearing clothing item 100. For example, sensor circuit 101 may contain a pressure sensor, an air temperature sensor, a humidity sensor, and an oxygen sensor. Sensor circuit 101 may also contain a wind speed sensor and solar radiation sensors which are installed at the location of measurements and which transmit data via a wireless signal to circuit 104. Sensor circuit 104 may contain a data logger and an expert system that processes and analyzes the collected data transmitted by the different sensors. Sensor circuits 102 and 103 may also include any one of the sensors listed for sensor circuit 101, including, for example, a speed sensor, a solar radiation sensor and so on. In one embodiment, sensor circuits 102 and 103 include pressure sensors to measure external pressure exerted on the individual wearing the clothing item 100 by, for example, a crowd.

FIG. 2 is a sample sensor configuration within a clothing item according to an exemplary embodiment. Clothing item 100 may also include sensor circuit 105. Sensor circuit 105 may include a wide array of sensors, including, but not limited to, humidity and oxygen sensors and pressure sensors. In one embodiment, sensor circuit 105 includes pressure sensor to measure the pressure of the crowd exerted on the individual wearing clothing item 100 from the back side. Sensor circuit 105 and all other sensors 101, 102 and 103 may be strategically placed anywhere within clothing item 100 in order to maximize the effective capture of relevant data. For example, sensor circuit 105 may be placed in the rear of the clothing item 100 in order to better capture a pressure exerted from a back location. Alternatively, if sensor circuit 105 included solar or oxygen sensors, then it may be strategically placed in a different location in order to better capture data related to sun exposure or to capture on-ground oxygen levels experienced by an individual wearing the clothing item 100. Each sensor circuit 101, 102, 103 and 105 may include more than one sensor to measure a wide array of parameters. For example, sensor circuit 105 may include a pressure sensor as well as a solar radiation and an oxygen level sensor. Of course in other embodiments, different combination of parameter capturing sensors may be included in any one of the sensor circuits 101, 102, 103 and 105 as one of ordinary skill in the art would recognize.

Referencing FIG. 1 again, clothing item 100 may also include a data logger and an expert system 104 that can communicate with sensor circuits 101, 102, 103 and 105 to process and manipulate the data received from the sensor circuits. In one example, the data logger may log all data captured by the sensor circuits 101, 102, 103 and 105 and categorize them in specific categories. Sensor circuits 101, 102, 104 and 105 are not the only possible configuration for any number of sensors that may be placed within clothing item 100. For example, clothing item 100 may include a fewer amount of sensor circuits that are designed to capture greater amount of data. Alternatively, clothing item 100 may include a greater amount of sensors that have specific tasks of capturing at least one parameter and may further be strategically positioned to capture that parameter. As previously mentioned, a solar sensor may be placed in a strategically specific location to capture sun radiation data, and alternatively, an oxygen sensor may be placed in a different strategically specific location. Thus, FIG. 1 is merely exemplary rather than limiting in the present disclosure.

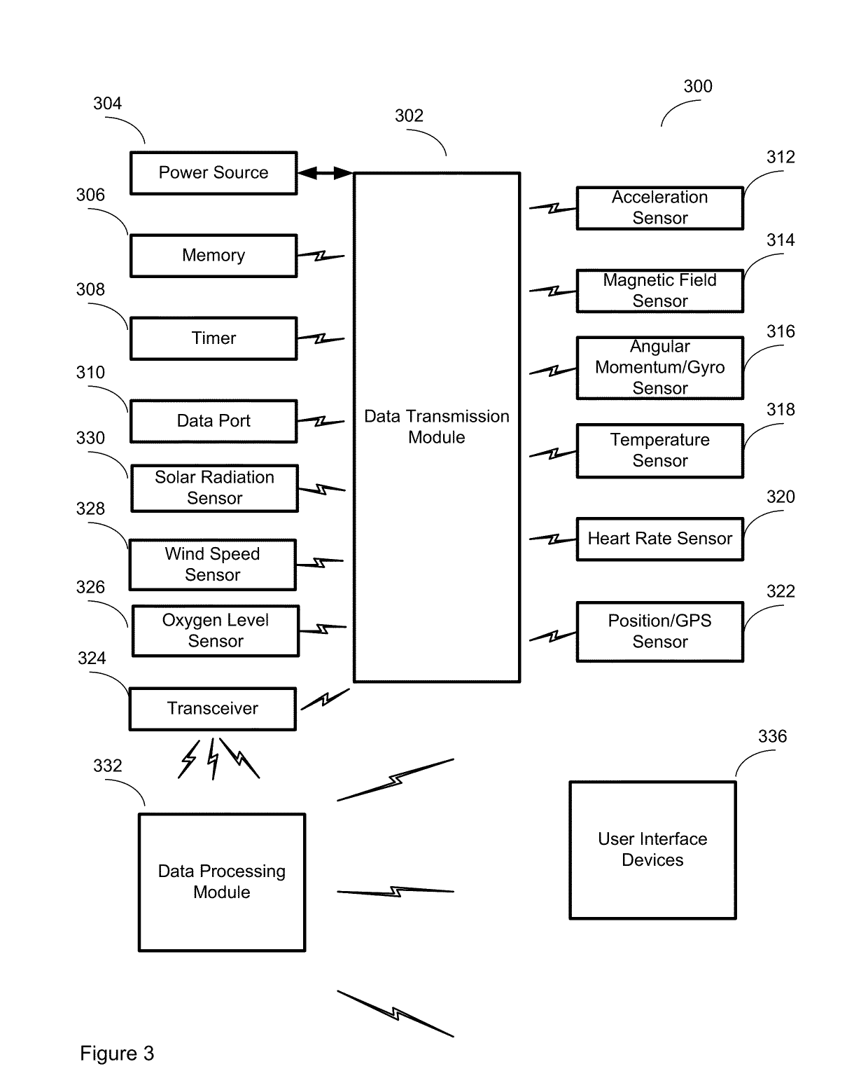

FIG. 3 is a system overview of a data logger and transmission and pre-processing systems within the comfort level monitoring system 300 and a given interaction configuration with different sensor blocks according to an exemplary embodiment. To monitor the comfort level of the individual wearing clothing item 100, in real-time scenarios, a comfort level monitoring system effectively monitors different types of discomfort, pressures and activities of every individual wearing clothing item 100 and communicate the data to a central computer for complete analysis. Data transmission circuit 302 collects and processes such information. Data transmission circuit 302 may be placed as yet another sensor on clothing item 100, as part of a sensor assembly on the individual (e.g., sensor block 104) or may be placed remotely. Data transmission circuit 302 includes a power source 304, memory 306, timer 308, data port 310, acceleration sensor 312, magnetic field sensor 314, angular momentum/gyro sensor 316, temperature sensor 318, heart rate sensor 320, position/GPS sensor 322 and transceiver 324, oxygen level sensor 326, wind speed sensor 328, and solar radiation sensor 330.

The power source 304 may provide power to the data transmission circuit 302. In one embodiment, the power source 304 may be a battery. For example, the power source may be built into the data transmission circuit 302 or removable from the data transmission circuit 302, and may be rechargeable or non-rechargeable. In an exemplary embodiment, the power source 304 may be recharged by a cable attached to a charging source, such as a universal serial bus (“USB”), FireWire, Ethernet, Thunderbolt, or headphone cable, attached to a personal computer. The power source 304 may also be recharged by inductive charging, wherein an electromagnetic field is used to transfer energy from an inductive charger to the power source 304 when the two are brought in close proximity, but need not be plugged into one another via a cable. A docking station may also be used to facilitate charging. The data transmission circuit 302 may further be repowered by replacing one power source 304 with another power source 304.

The memory 306 may store application program instructions and store athletic activity data. In an embodiment, the memory 306 may store application programs used to implement aspects of the functionality of the comfort level monitoring system described herein. The memory 306 may also store raw data, recorded data, and/or calculated data, and/or as explained in farther detail below, the memory 306 may act as a data storage buffer. The memory 306 may include both read only memory and random access memory, and may further include memory cards or other removable storage devices. The memory 306 may store data in memory locations of predetermined size such that only a certain quantity of data may be saved for a particular application of the current data transmission circuit 302.

The timer 308 may be a clock that tracks absolute time and/or determines elapsed time. In some exemplary embodiments, the timer 304 may be used to timestamp certain data records, such that the time that certain data was measured or recorded may be determined and various timestamps of various pieces of data may be correlated with one another.

The data port 310 may facilitate information transfer to and from the data transmission circuit 302 and may be, for example, a USB port. The data port 310 can additionally or alternatively facilitate power transfer to power source 304, in order to charge power source 304.

A hydration sensor, not shown, may be provided in the clothing item 100 to measure the individual's hydration parameters. The hydration sensor may further include elements such as a tilt sensor on a beverage dispenser, for example, a water bottle, that make a near field communication connection with the data logger to provide a measurement of how many sips of a drink the individual is taking. This information can be compared to hydration levels and environmental conditions, such as temperature and humidity, to provide a coded or tactile alert to the wearer of clothing item 100 or provide an alarm message to the monitoring system if the individual or crowd is being detected as becoming dehydrated. The monitoring system may interpret a single individual as a representative sample of a crowd or may alternatively deploy multiple clothing items of clothing item 100 to generate a greater sample size.

The acceleration sensor 312 may measure the acceleration of the data transmission circuit 302 when it is placed on the individual or the acceleration of sensors on clothing item 100 worn by the individual. For example, if data transmission circuit 302 is physically coupled to an object, such as clothing item 100 or any item placed on an individual (not shown), the acceleration sensor 312 may measure the acceleration of the individual, including the acceleration due to the earth's gravitational field. In one exemplary embodiment, the acceleration sensor 312 may include a tri-axial accelerometer that measures acceleration in three orthogonal directions. Of course two, three, or more separate accelerometers may be used in the alternative without departing from the scope of the present disclosure.

The magnetic field sensor 314 may measure the strength and direction of magnetic fields in the vicinity of the data transmission circuit 302. Accordingly, when the data transmission circuit 302 is physically coupled to an individual or clothing item 100, the magnetic field sensor 314 may measure the strength and direction of magnetic fields in the vicinity of the individual, including the earth's magnetic field. In one exemplary embodiment, the magnetic field sensor 314 may be a vector magnetometer. The magnetic field sensor 314 may also be a tri-axial magnetometer that measures the magnitude and direction of a resultant magnetic vector for the total local magnetic field in three dimensions. Two, three, or more separate magnetometers may be used as one of ordinary skill would recognize.

In one example, the acceleration sensor 312 and the magnetic field sensor 314 may be contained within a single accelerometer-magnetometer circuit integrated circuity such as LSM303DLHC made by STMicroelectronics of Geneva, Switzerland. The data logger may include only one of the acceleration sensor 312 and the magnetic field sensor 314, and may omit the other if desired.

The angular momentum/gyro sensor 316, which may be, for example, a gyroscope, may be adapted to measure the angular momentum or orientation of the data transmission circuit 302. Accordingly, when the data transmission circuit 302 is physically coupled to an individual or clothing item 100, the angular momentum/gyro sensor 316 may measure the angular momentum or orientation of the individual. The angular momentum/gyro sensor 316 may be a tri-axial gyroscope that measures angular rotation about three orthogonal axes. Two, three, or more separate gyroscopes may be used instead, however. In an exemplary embodiment, the angular momentum/gyro sensor 316 may be used to calibrate measurements made by one or more of the acceleration sensor 312 and the magnetic field sensor 314.

The temperature sensor 318 may be, for example, a thermometer, a thermistor, or a thermocouple that measures changes in the temperature. The temperature sensor 318 may be used for calibration of other sensors of the comfort level monitoring system, such as the acceleration sensor 312 and the magnetic field sensor 314. The temperature sensor 318 may also be used to calculate the energy expenditure of the individual wearing clothing item 100, and/or to calculate temperature and humidity levels of the surrounding environment. Such temperature and humidity levels can be further utilized to calculate environmental comfort level (ECL), as described in detail below.

The heart rate sensor 320 may measure the individual's heart rate and may be placed in contact with the individual's skin, such as on the chest or wrist, and secured with a strapping mechanism. Heart rate sensor 320 may provide an alarm signal if a person's heart rate falls below a given threshold or rises above a given threshold. For example, if a person's heart rate rises above 220 beats per minute (bpm) then the alarm signal is sent to the comfort level monitoring system 300. Alternatively, if a person's heart rate falls below 40 bpm, the alarm signal is also sent to the comfort level monitoring system 300. In another example, the predetermined threshold may be tailored for different personal attributes of the person wearing the sensor. For example, a different threshold may be designated for a man than for a woman. Alternatively, a different threshold may also be designated depending on age of the person and weight of the person.

The position/GPS sensor 322 may be an electronic satellite position receiver determines its location (i.e., longitude, latitude, and altitude) using time signals transmitted along a line-of-sight by radio from satellite position system satellites. Known satellite position systems include the GPS system, the Galileo system, the BeiDou system, and the GLONASS system. The position/GPS sensor 322 may also be an antenna that communicates with local or remote base stations or radio transmission transceivers to determine the location of data transmission circuit 302 using radio signal triangulation or other similar principles. The position/GPS sensor 332 data may allow data transmission circuit 302 to detect information that may be used to measure and/or calculate position waypoints, time, location, distance traveled, speed, pace, or altitude as would be recognized by one of ordinary skill.

The transceiver 324 may enable the data transmission circuit 302 to wirelessly communicate with other components of the comfort level monitoring system 300, such as those described in further detail below. For example, the transmission circuit 302 and the other local components of the comfort level monitoring system 300 may communicate over a personal area network or local area network using, for example, one or more of the following protocols: ANT, ANT+ by Dynastream Innovations, Bluetooth, Bluetooth Low Energy Technology, BlueRobin, or suitable wireless personal or local area network protocols. Other known communication protocols suitable for a comfort level monitoring system 100 may also be used.

In one exemplary embodiment, transceiver 324 is a low-power transceiver and may be a two-way communication transceiver 324, or a one-way transmitter or a one-way receiver. Wireless communication between data transmission circuit 302 and other components of the comfort level monitoring system 300 is described in further detail below. Alternatively, the data transmission circuit 302 may be in wired communication with other components of the comfort level monitoring system 300 and not rely on transceiver 324 as can be appreciated.

Other sensors may also be included in the system and may communicate with data transmission circuit 302. For example, the system may include oxygen level sensor 326, wind speed sensor 328 and solar radiation sensor 330.

Oxygen level sensor 326 may measure oxygen levels in the surrounding areas where the individual is conducting a physical activity such as walking, running, hiking, or sitting. Oxygen level sensor 326 may be further used to measure oxygen level ratios within the surrounding environment, and may compare a ratio of existing oxygen levels to expected or normal oxygen levels for that environment. Oxygen level sensor may be any type of oxygen level sensors, including, for example, Honeywell oxygen sensor having 3 series of flange mounts and probe housing that contains two Zirconium Dioxide (ZrO2) discs with a small, hermetically sealed chamber between each disc. The ZrO2 technology provides oxygen measurement without reference gas, which results in enhanced accuracy and durability.

Wind speed sensor 328 may be an anemometer used to measure wind speed at a given location. Diverse types of anemometers may be deployed depending on their location of deployment. For example, cup and vane anemometers may be difficult to deploy within clothing item 100, but may be deployed throughout the geographic vicinity where the individual and crowd are expected to be gathered. A single anemometer may be used, or alternatively, a series of anemometers are used, each providing potentially different wind speeds for different locations to the comfort level monitoring system 300. A hot wire anemometer may further be deployed within clothing item 100. A hot wire anemometer uses a very fine wire electrically heated up to some temperature above the ambient. Air flowing past the wire has a cooling effect on the wire. As the electrical resistance of most metals is dependent upon the temperature of the metal, a relationship can be obtained between the resistance of the wire and the flow speed. Of course, other anemometer types may also be used without departing from the scope of the present disclosure. Thus, the anemometers described herein are merely exemplary and not limiting upon the disclosure.

Solar radiation sensor 330 may determine the available solar energy radiation and may include a light sensing element that detects the quantity or intensity of solar radiation and converts the quantity or intensity into an electric signal. The electric signal is sent to data transmission circuit 302 to determine solar radiation exposure and intensity, which is a parameter that may be used in determining the comfort level of the individual. An example of a solar radiation sensor can be a pyranometer that is used to measure the solar radiation flux at a given time from a field of view of 180 degrees. The pyranometer does not require any power to operate, thus reducing the potential energy consumption of the comfort level monitoring system 300.

Once data transmission circuit 302 collects all sensor data associated with an individual or a group of individuals, the data may be forwarded to data processing circuit 326. Data processing circuit 326 may process and house a variety of information for dissemination to multiple user interface devices such as devices 336 as will be further described below. Data processing circuit 326 may be a part of the data transmission circuit 302, or may be integrated within a server or a central server that performs certain processing functions, such as processing the data received from data transmission circuit 302. As can be appreciated, the data processing circuit 326 may include the necessary circuitry to perform the above described functions. Such circuitry may be, for example, a processor, micro controller, logic device, or any combination of these as would be recognized by one of ordinary skill in the art.

Comfort level expert system 300 100 may further include features that enable the data processing circuit 326 to interact with web based systems to retrieve individual related statistics. For example, data processing circuit 326 may connect to and process data from a single data transmission circuit or a specified number of data transmission circuits associated with a given number of individuals. These individuals can be placed in groups or can be randomly deployed to determine comfort levels in different environments and at different locations within given geographic vicinity. However, in order to perform specific data manipulations and comparisons on a geographic vicinity wide basis, data processing circuit 326 may connect to internet based systems to download other individual/group information. This is useful in determining location-based statistics within specific geographic vicinity. In one example, data processing circuit 326 may communicate with multiple data transmission circuits deployed on individuals in a concert venue. Different data transmission circuits may be utilized to indicate different comfort levels for individuals in different sections of the concert venue, such as a park or an arena. For example, one data transmission circuit may be located within the floor area, another may be located directly by the stage area and a third may be located in specific sold out sections, alleys, walkways, etc.

In yet another example, individuals with data transmission circuits may be deployed during a Hajj season, such that different sections of Mecca may be monitored for crowd comfort levels and crowd safety controls. During Hajj and Ramadan, the holy shrines in Mecca witness greater influx of visitors. The increase in crowd densities during the religious ceremonies (known as Tawaf) can raise great concerns regarding individual comfort levels within the crowd. For example, during Hajj, more than 4 million visitors visit the holly sites in Mecca. This great number of people greatly affects individual comfort levels within crowds.

Furthermore, due to geographic and architectural attributes of the holy sites, there will be locations where crowds may “bottle neck” or simply gather in greater numbers while awaiting their turn to perform the religious functions. The comfort level monitoring system enables monitoring of individual comfort levels within a crowd, but also helps administrators determine crowd behavior, crowd movement and how to best address overcrowding problems. For example, in one situation, an administrator monitoring an individual within a crowd may receive alarm signals indicating great discomfort for that person. The personal comfort dynamic will likely also apply to the crowd as people within close proximity to the individual will likely feel the same discomfort. The administrator can use this information to abort the activity, take additional measures for enhanced crowd movement/control, and also make structural design adjustments to alleviate observed high pressure areas.

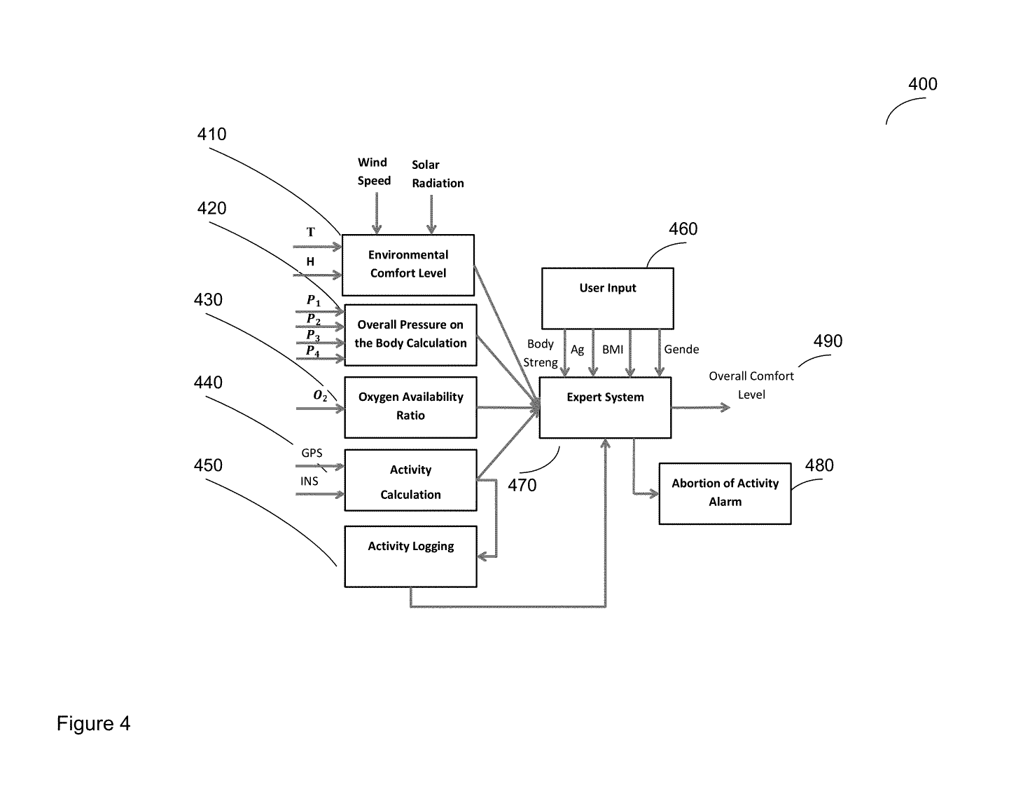

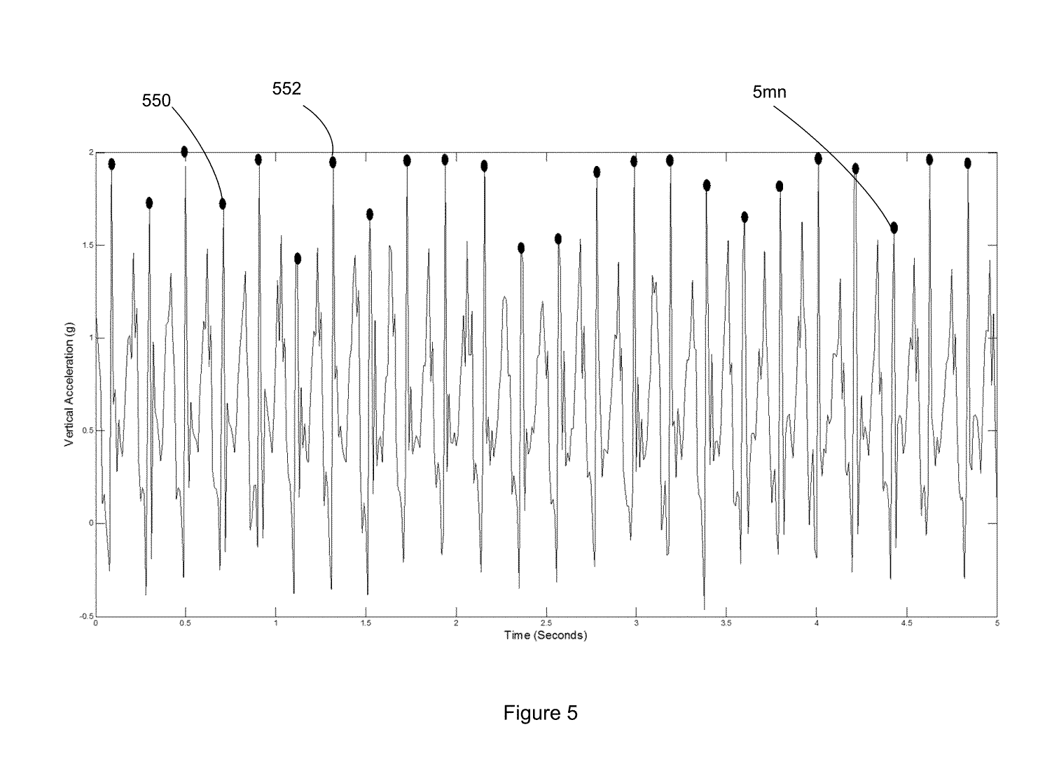

Every person has his normal walking speed on which he feels comfortable. If his walking speed becomes slow due to encountering a crowd, he may feel stress and discomfort. His discomfort increases as he is forced to keep his walking speed reduced for longer duration of time. Hence, activity recognition and walking speed estimation is also an important factor contributing to the comfort level. FIG. 4 is a system overview of a comfort level expert system 400 according to one an exemplary embodiment. The comfort level expert system 400 includes an environmental comfort level aggregating circuitry 410, an overall pressure on the body calculating circuitry 420, oxygen availability ratio measuring circuitry 430, activity calculating circuitry 440, activity logging circuitry 450, user interface circuitry 460, expert system 470 and an activity abort alarm system 480.

Environmental comfort level aggregating circuitry 410 is designed to take in a multitude of inputs and aggregate an output that is later fed into the expert system 470. Environmental comfort level aggregating circuitry 410 is designed to take in several parameters, including, for example, wind speed, solar radiation, temperature and humidity and aggregate an output in a non-linear fashion. Instead of taking each input into account, separately and adding them together, the environmental comfort level aggregating circuitry 410 creates an n dimensional space, n being an integer equivalent to the inputs into the environmental comfort level aggregating circuitry. In one example, n can be 4, and creates a dependency of evaluation. Instead of creating a linear addition of 4 individual dimensional spaces, a linear combination of two 2-dimensional can be generated. A four dimensional space can also be generated creating an inter dependency between the four inputs such that that the effects of each individual one-dimensional space are also calculated on all other dimensional spaces within the four dimensional space. This creates a more enhanced determination of the factors such that wind, for example, is not looked at separately, but instead, as part of a four dimensional space of interdependent parameter inputs that all affect each other.

Environmental comfort level aggregating circuitry 410 calculates environmental comfort level using neural network as regression optimization or function approximation. For example, the relationship between inputs and outputs of environmental comfort level aggregating circuitry 410 is a non-linear relationship because a neural network as a universal function approximation device is used. For example, with the same level of temperature and humidity, the comfort level will be different if a person is in the shade or standing in the sun as would be indicated by, for example, solar radiation sensor 330. Similarly, wind speed may produce a cooling effect in case of low humidity and this will also affect the comfort level. In an exemplary embodiment, training examples are collected from subjective judgments of the individual wearing the clothing attire 100, and as such, a database that is stored in memory 302 is installed for housing training examples from many individuals to be used to train the neural networks.

Overall pressure on the body calculating circuitry 420 generates an input that takes into account pressure values transmitted from pressure sensor circuits dispersed throughout attire worn by an individual. For example, pressure inputs P1-P4 can be generated from sensor circuits 101, 102, 104 and 105 as illustrated from FIGS. 1 and 2. Alternatively, pressure sensor circuits can be distributed in many different allocations that can best capture the overall pressure exerted on an individual.

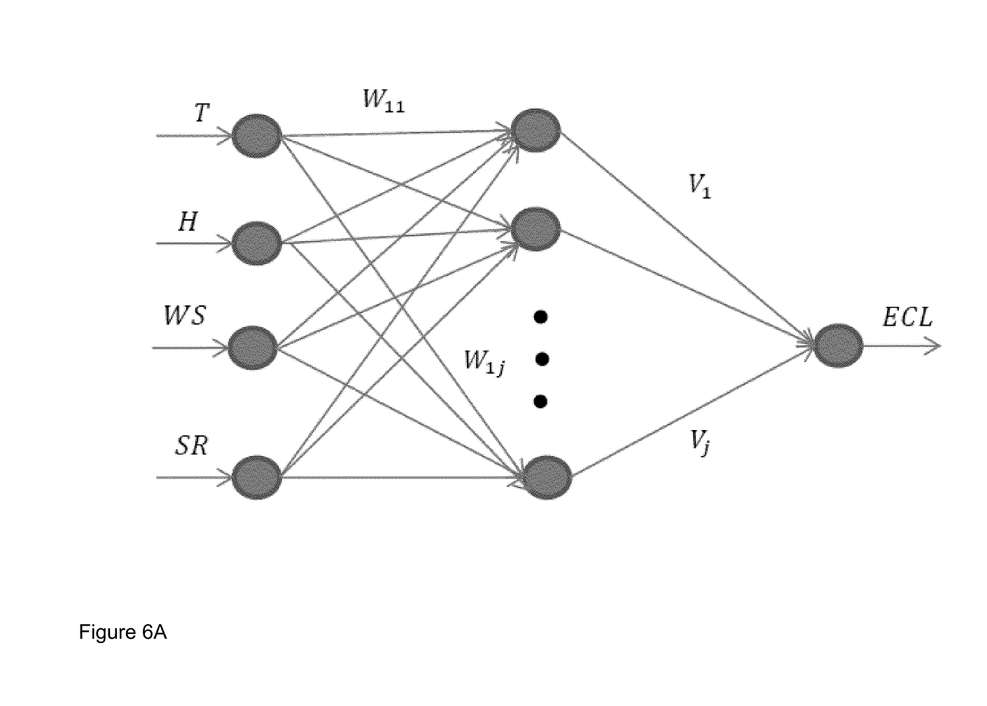

Crowd forces play an important role in disasters. Deaths in the crowd may be due to compressive asphyxia, which is lack of oxygen due to crowd pressure. Crowd forces pile up due to pushing of the people or people leaning against each other. Hence, it is important to calculate the crowd forces on the person in the crowd. In this regard, the four pressure sensors 101, 102, 103 and 105, or P1-P4 in FIG. 5, can be placed on the clothing item 100 to record the crowd forces on the body. Pressure sensors on the chest and the back can predict the compressive asphyxia that may lead to suffocation. Similarly, pressures on the sides of the person may cause the person to rotate his/her orientation or cause the person to fall down. In a crowd of five people, a compressive force of up to 3,430N can be developed. Tolerable compressive forces for men are typically up to 623N, and compressive forces of 6,227N for 15 seconds can be lethal to a person. As such, pressure values from all sensors are summed up to calculate an overall pressure, OP, on the person's body.

$$OP = {{\sum }^{4}}_{i = 1} {β}_{i} {P}_{i} \qquad Equation ( 1 )$$

In equation 1, βₜ are the weights associated with the different location of the pressure sensors Pₜ. Front and back sensors are given more weight as compared to the sideways pressure sensors. However, other weight distributions are possible as one of ordinary skill would recognize

Oxygen availability ratio circuitry 430 measures the available oxygen levels measured by an oxygen sensor, such as oxygen level sensor 326 that surrounds the individual. Oxygen availability ratio is the ratio by which oxygen content is measured as a percentage of air samples measured around the individual. The ratio may have a threshold such that if the oxygen availability ratio falls below the threshold then expert system 470 takes the below threshold value into account as it calculates comfort levels.

Concentration of oxygen in the atmosphere is 20.9% of the total gases. Oxygen deficiency in the atmosphere may cause some serious effect on the condition of human being. If the concentration of oxygen falls to 14%, for example, abnormal fatigue and emotional upset may result. Oxygen concentration less than 10% can be lethal if not restored immediately. Hence it is important to know the available oxygen ratio in the atmosphere. Oxygen ratio is calculated as:

$$OR = \frac{Oxygen\_con ( \% age )}{21 \%} \qquad Equation ( 2 )$$

Whenever oxygen is above certain threshold, for example, OR≧1, OR factor will not be taken into consideration in the comfort level calculations. However, when oxygen deficiency occurs, this becomes a problem that will contribute in the discomfort of the person. As previously described, OR levels below a threshold can contribute to discomfort and can further become life threatening. As such, OR levels below predetermined thresholds contribute in the comfort level calculations and may further require an abortion of activity alarm to be activated. Equation 2 illustrates a calculation for the oxygen ratio. Oxygen_con(% age) is the atmospheric concentration of the oxygen which is divided by the normal oxygen concentration in the environment (˜21%). Oxygen_con(% age) can be measured by, for example, oxygen level sensor 326. As such, if OR is greater than or equal to a value one, then the conditions are deemed to be normal conditions. As OR values begin to fall below on, i.e. lower atmospheric concentrations, the violation of the threshold will be a contributing factor in aborting the activity via designated alarm mechanisms.

Activity calculation circuitry 440 takes into account multiple inputs, including the global positioning system (GPS) information of the individual as well as inertial navigation system (INS) information of the individual. The INS information can be generated from a multitude of sensors, including motion sensors such as acceleration sensor 312 (accelerometer) and rotation sensors or angular momentum/gyro sensor 316 (gyroscope). The INS can be useful to continuously calculate, via dead reckoning, the position, orientation, and velocity (or direction and speed of movement) of a moving person without the need for external references. INS data can be used by itself to determine individual movement data or in collaboration with GPS information. In one embodiment, where both INS and GPS inputs are used to measure the location and activities of the individual, the INS data can be used to help calibrate GPS data for increased accuracy.

Activity calculation circuitry 440 may further include data analysis to extract features associated with the individual. For example, feature extraction may include features and motions associated with specific activities, i.e., a walking activity may be associated with a different activity than a standing or running activity. As such, feature extraction allows for extraction of all relevant physical features associated with the individual. Activity calculation 440 includes classifying motions and movements of the individual into specific activities. Such activities include, but are not limited to, walking, running, sprinting, standing, jogging, and so on. The classified activities are recorded in real time and passed on for additional data analysis.

As part of the activity calculation circuitry 440, the comfort level expert system 400 also includes activity logging circuitry 450. Activity logging allows for each activity that is calculated to be logged in a system memory, such as memory 306 for further downstream analysis. Activity logging circuitry 450 may enable data analysis that includes modifications of system tolerances, calibration of ratios that may yield different comfort levels, or data tracking for enhanced event planning/management. Expert system 470 may have the option to retrieve such data from activity logging circuitry 450. Activity logging circuitry 450 may be performed by a multitude of processing capabilities within the system, including data transmission circuit 302 or data processing circuit 332.

In yet another exemplary embodiment, activity classification and logging circuitries are used to calculate the energy expenditure of the person, which also contribute to the comfort level prediction. Walking speed of the person can be calculated from GPS sensor if its signal is available. In case of GPS unavailability, cadence can be calculated from the INS information as previously described. In one example, cadence (walking steps per minute) is calculated by counting the maximum peaks of the acceleration in vertical acceleration and anterior/posterior acceleration. This example can be further shown in FIG. 5. Cadence can predict the walking speed as the stride length of the person follows a normal distribution with a mean value. This mean value of the stride length is used to calculate the walking speed. For example, FIG. 5 includes peaks 550, 552, and 5mn wherein (mn) are integers indicating a number of peaks that help generate a stride length and walking speed parameters.

To further aid the expert system 470 in producing a more accurate overall comfort level 490, expert system 400 may utilize user inputs 460 as additional parameters. The user inputs 460 are designed to maximize accuracy and take into account appropriate information related to the individual. Such information includes, but is not limited to, body strength information, age, body mass index (BMI) and gender information to be input into the expert system 470. Other inputs may also be useful to the expert system 470, including for example, individual's high, diet, etc. Such information can help refine the analysis of overall comfort level 490. For example, a man with certain physical attributes may feel different levels of discomfort than a woman with a different set of physical attributes, or two individuals having the same gender but different ages may further experience discomfort at different levels. Furthermore, two individuals with different BMI levels may also experience different levels of discomfort.

In one exemplary embodiment, taking an individual male as an example, every person has his normal walking speed which feels comfortable to them. If his walking speed slows due to encountering a crowd, he may feel stress or discomfort. His discomfort increases as he is forced to keep his walking speed reduced for longer duration of time. Hence, activity recognition and walking speed estimation is also an important factor contributing to the comfort level. Activity calculation circuit 440 provides the information of the activity to the expert system 470 and activity logging circuit 450 generates the information about the time record of a particular activity. As iterated previously, time record of a particular activity and activity logs may help downstream analysis of the individual activity, system and enhance calibration capabilities.

Expert system 470 utilizes rule based decision making operations based on the information received and comfort level of the person is calculated at a particular instance. In one embodiment, if the comfort level drops below certain predefined threshold, activity abort alarm 480 is initiated suggesting the user to abort the activity or try to escape from the crowd to avoid any discomfort or injury. Crowd based comfort level is an index that takes into account the values of overall pressure on the body and available oxygen level ratios. Personal cumulative activity index is an index related to the activity of the person, which can be calculated from the inertial sensor. These activities may include, as previously mentioned, walking, running, jogging, sprinting and standing. Cadence of the person can be calculated from the peak detection of the acceleration data in the y-axis and z-axis acceleration.

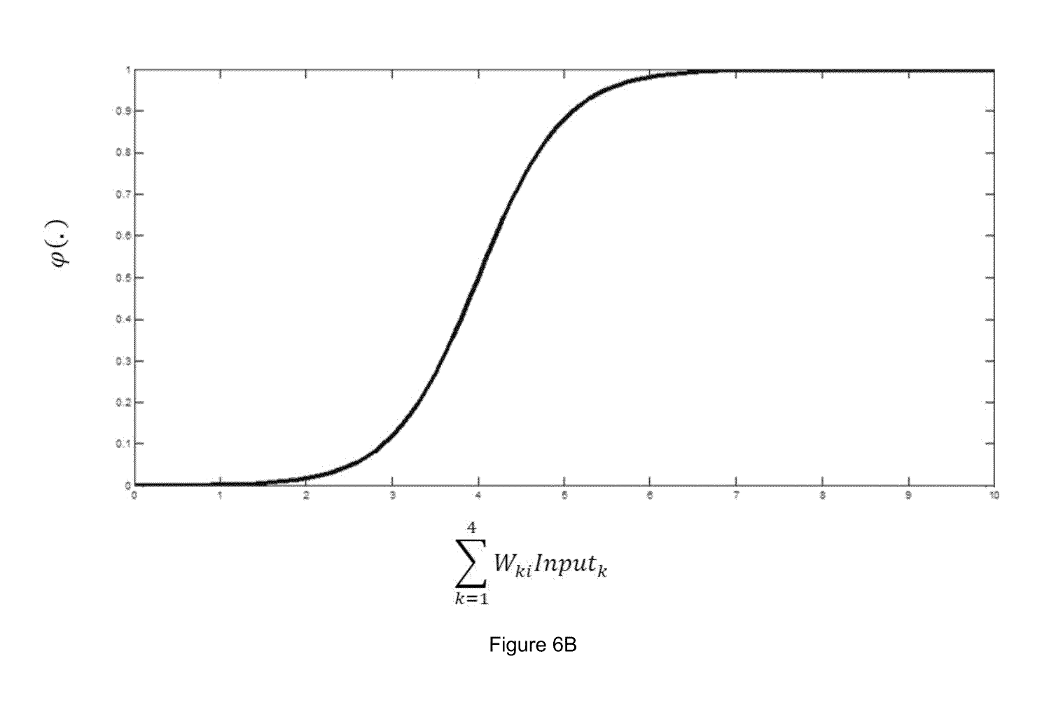

FIGS. 6A and 6B illustrate sample environmental comfort level (ECL) calculations and plotting according to exemplary embodiments. In FIG. 6A, the environmental comfort level is calculated by using two layer back-propagation neural network. In one example, the inputs of the neural network are temperature (T), humidity (H), wind speed (WS), and solar radiation level (SR). The output of the neural network may be calculated as:

$$ECL = φ ( {\sum }_{i = 1}^{j} {V}_{i} ϕ ( {\sum }_{k = 1}^{4} {W}_{ki} {Input}_{k} ) ) \qquad Equation ( 3 )$$

Wherein here φ(.) and φ(.) are the activation functions. An activation function maps the weighted sum of the inputs to an output value on a particular scale. The neural network is two layered network. First is the input layer which has four neurons (corresponding to H, T, SR, WS), second layer (corresponding to W₁ⱼ) is a hidden layer containing l neurons and the third layer is the output layer containing one neuron and output is ECL. As such, Inputₖ in Equation 3 corresponds to the four inputs to the neural network. For example T can be Input′, H may be Input₂, WS is Input₃ and SR is Input₄. The inputs are summed together by multiplying certain weights Wₖᵢ here k is counter for input (k=1 to 4) and i is the counter for the hidden neurons (total hidden neurons are l). Then φ(ΣΣₖ₌₁⁴WₖᵢInputₖ) is calculated. Here φ(.) is a nonlinear or linear activation function applied to the summation. The activation function can be linear, sigmoidal, tan h, or step function. For example, FIG. 6B illustrates a sigmoidal function output reflecting the selection of the activation function φ(.) to generate a variable output from 0 to +1. In one example, the activation function is used for the approximation of the nonlinear function. Nonlinearity generated from the activation function contributes in approximating the nonlinear functions. Furthermore, the activation function also helps to control the large values of summations to maintain within certain limits.

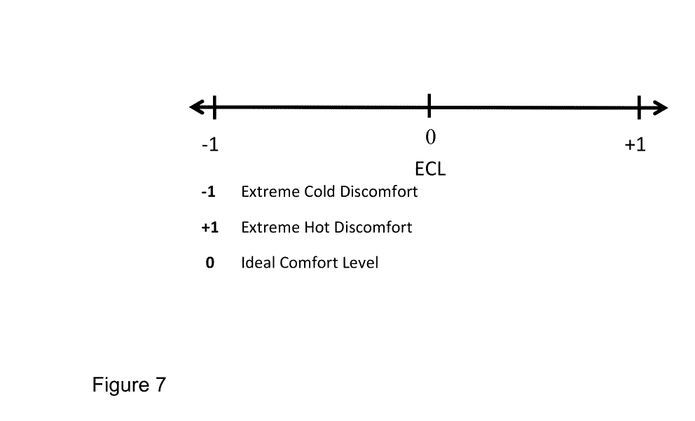

FIG. 7 is an environmental comfort level scale to determine comfort or discomfort levels according to an embodiment. As illustrated in FIG. 6, the discomfort levels range from (−1) to (+1) indicating that (−1) is an extreme cold discomfort value and (+1) is an extreme hot discomfort value, wherein a (0) value indicates an ideal comfort level. As such, values between (0) and (+1) indicate a progressive hot discomfort indication up to a (+1) extreme hot discomfort indication. Similarly, values between (0) and (−1) indicate a progressive cold discomfort indication up to a (−1) extreme cold discomfort indication. Environmental discomfort comes from different factors. A primary weight is given to temperatures. Other parameters like humidity, solar radiation and wind speed are used to modulate the discomfort level further to (+1) or further to (−1) depending on the temperature. For example, (−1) will be corresponding to the discomfort when temperature is very low, e.g. below freezing, while (+1) corresponds to the discomfort related to high temperatures, e.g. above 45 degree Celsius. The neural network are trained in a manner that produces negative values for the discomfort due to cold and produces positive values for the discomfort due to hot weather.

Environmental comfort level (ECL), Overall pressure (OP) and horizontal pressure (HP) on the body, available oxygen ratio and personal cumulative activity index is fed to the expert system which is a fuzzy expert system. Similarly age, BMI and body strength indicator is also fed to the expert system. Based on the fuzzy expert system, overall comfort level may be calculated.

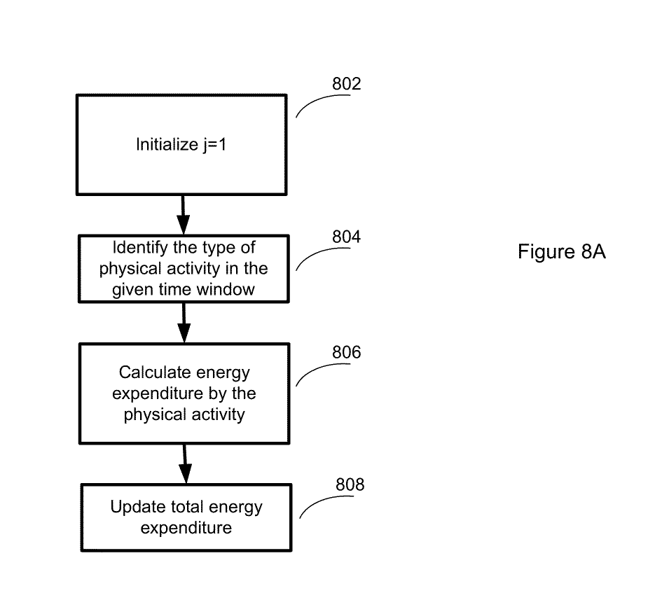

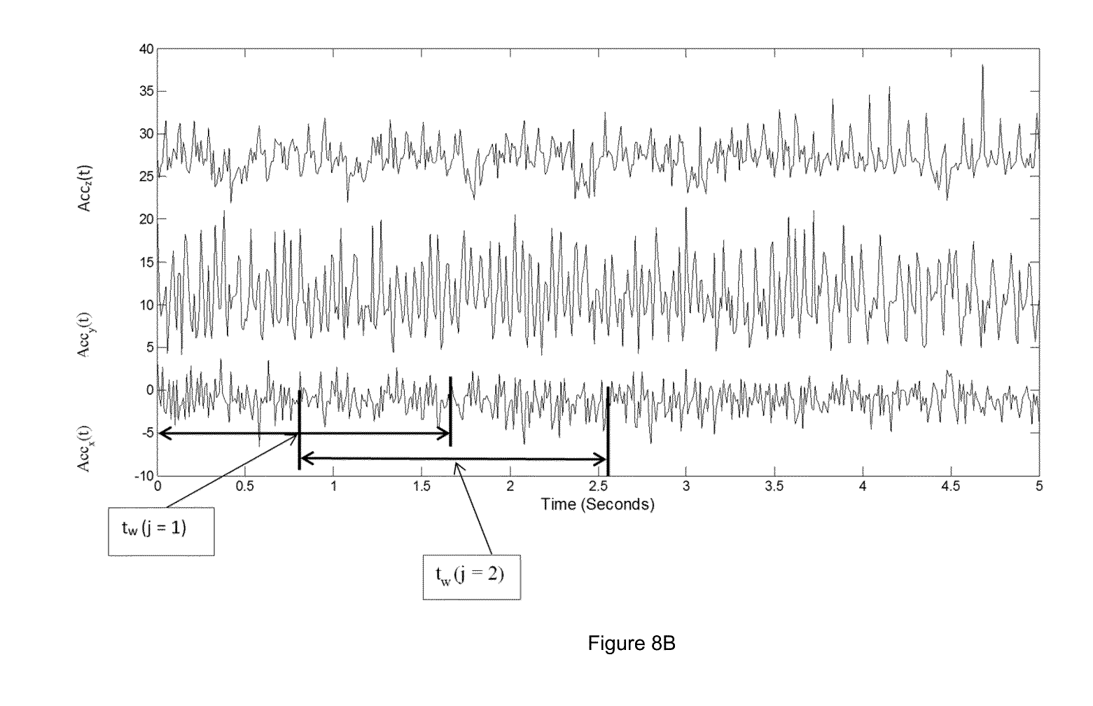

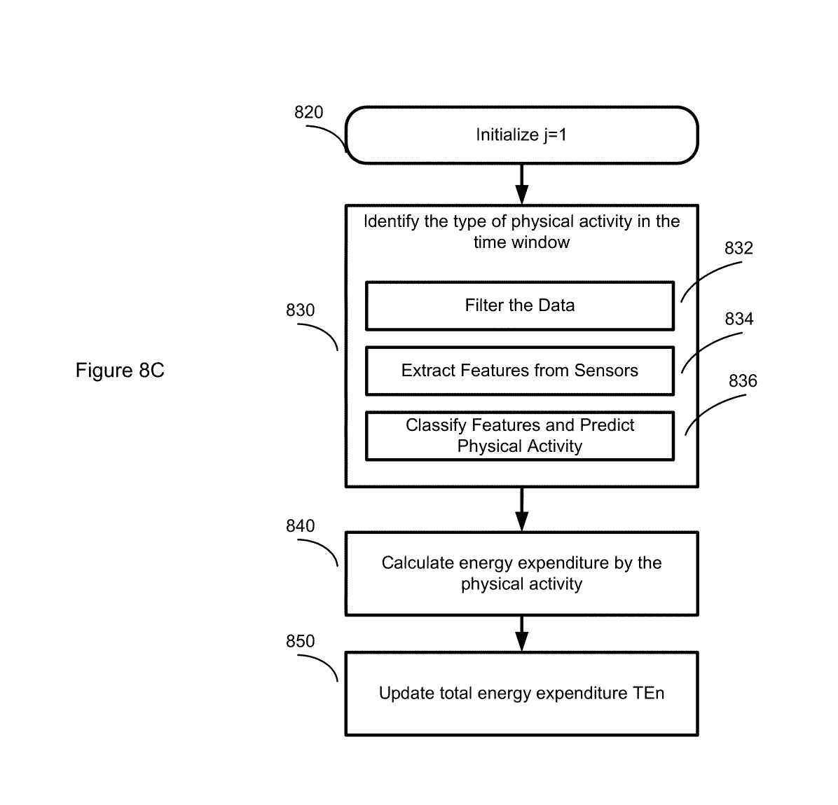

FIG. 8A is a sample activity logging and calculation algorithm according to one exemplary embodiment. As an initial matter, the value j, which is a time interval, is initialized to a value 1 at step 802. Time interval j can be set to any time window value. For example, time interval j=1 can be a time window into a 2 second capture of acceleration and GPS data as a function of time. The algorithm further includes identifying, 804, the type of physical activity in the given time window. The physical activity can be any type of activity, including, but not limited to, standing, walking, running, sitting, sprinting, etc. This activity is typically the i^{th} physical activity of the individual at time t. The algorithm further includes calculating, 806, energy expenditure by the physical activity conducted by the individual and updating, 808, the total energy expenditure.

FIG. 8B illustrates activity acceleration and GPS plot as a function of time according to an exemplary embodiment. A time window t_{w} is defined and is assigned a segment of acceleration and GPS data. FIG. 8B illustrates 3 time domain signals of acceleration data for an activity carried out by the individual wearing clothing attire 100. For example, Accₓ(t) can be acceleration and GPS data plot of an activity from a particular sensor, while Acc_{y}(t) can be acceleration and GPs data plot of a different sensor. In one example, a physical activity being performed is determined using a time window methodology. Here, at separate intervals, j=1, 2, 3 . . . , n, time windows t_{w} are captured. An algorithm is applied to each t_{w} to determine the physical activity. In one application, t_{w} is a sampling method used to sample the activity at sequential intervals, such that at every interval, a time window is captured and a physical activity classification algorithm is applied to determine the physical activity being performed. Physical activities may include, but are not limited to, walking, running, jogging, etc.

A further illustration of the physical activity classification algorithm is illustrated in Table I below.

TABLE I
Algorithm of Activity Classification and Logging
Definitions:

| | |
|---|---|
| PA | Physical activities Standing, Walking, Running, Sitting |
| PAᵢ (t) | i^{th} Physical Activity of the player at time t |
| t | Time variable |
| t_{w} | Time window to calculate the physical activity |
| TEn | Total Energy expenditure |

Main Algorithm
Step 0: Initialize j = 1
Step 1: Identify the type of physical activity in the time window,
PAᵢ (j) = Identify_PA(Acc(t_{w}), GPS(t_{w}), Gyro(t_{w}))
Step 2: Calculate Energy expenditure by the physical activity
En^{t}^{_{w}} = Calculate_Energy_Expenditure(PAₜ(j), t_{w})
Step 3: Update total energy expenditure TEn = TEn + En^{t}^{_{w}}
Physical Activities Classification Algorithm
Identify_PA(Acc(t_{w}), GPS(t_{w}), Gyro(t_{w}))
Acceleration, gyro and GPS signals are recorded by the sensors placed on
the jacket. Before calculating any feature, the raw accelerometer and gyro
data was preprocessed to reduce noise using median filter or order n in
each dimension separately. A window of wₜ seconds (fₛ × wₜ samples)
is used to calculate the feature set for a particular activity.
Here, fₛ is the sampling frequency of the data.
Step 1: Pre-processing of the data through filter.
Step 2: Appropriate feature extraction from the sensors data.
Step 3: Classification of the features and prediction of the physical
activity.

FIG. 8C illustrates a sample activity classification algorithm according to an exemplary embodiment. As an initial step, the algorithm reflected in Table I initializes a time window increment to a value j=1. For example, a first time window may be 0-1 seconds, which will be denoted as j=1. A second time window may be 1-2 seconds, which will be denoted as j=2, and a third time window may be 2-3 seconds, which will be denoted as j=3, and so on. The algorithm further includes identifying, 830, the type of physical activity in the time window (j=1, 2, . . . , n). In one example, physical activity is identified by firstly filtering the sensor data outputs 832, then extract features from sensors 834 and followed by classifying features and predicting physical activity 836. In the activity classification algorithm, identification of the type of physical activity will be done by the following steps:
Step 1: From the captured time window signal of acceleration and gyro sensor, time domain features will be calculated (time domain features may include simple features like mean absolute value, harmonic mean, variance, root mean square, skewness, Kurtosis, cumulative length zero, crossing rate, slope sign change, simple squared integral, correlation coefficient, etc.)
Step 2: An appropriate classifier, such as a random forest classification method is used to identify the type of activity.

GPS signal is used as to help improve the classification and to find out the distance covered. Energy expenditure is calculated by the amount of time a person is performing the physical activity and what is the type of activity. A parameter which explains the intensity of the physical activity is used, such as Metabolic Equivalent Task (MET). For example, one MET is the energy expended at rest and establishes that value as a baseline for further energy expended, such as two METs, three METs and so on. A three METs energy expenditure is equivalent to three times the rest energy expended at resting position. A MET table can be used, which associates different activities with MET values. For example, standing is associated with a (MET=1.3), slow walking is associated with (MET=2.0), fast walking is associated with (MET=2.5) and so on. MET values for each activity are approximations and may have individual variation as well depending on the body mass and age of the person, and as such, do need to be adjusted. Energy expenditure in Kcal (kilo calories) is then calculated from MET level, age and body mass levels by using an appropriate formula or regression. To calculate a total energy expenditure, the energy expenditure by each physical activity is calculated 840 and then a total energy expenditure 850 is calculated.

In one exemplary embodiment, sensor circuits (not shown) may be further incorporated in clothing items worn on an individual's thighs and waist, such as shorts, trousers, pants, and belts, to further measure compressive pressure on the individual. Additionally, the sensor circuits can be used to measure acceleration signals that are also recorded by the sensors placed on thighs and waist. Before calculating any feature, the raw accelerometer and gyro data is processed to reduce noise using a filter, such as a median filter or order n filter, in each dimension separately.

A Fuzzy expert system is an expert system that uses a collection of fuzzy membership functions and rules, instead of Boolean logic, to reason about data. The fuzzy set theory deals with reasoning that is approximate rather than precisely deduced from classical predicate logic such as Boolean logic. The rules in a fuzzy expert system are usually of a form similar to the following: if x is low and y is high then z=medium; where x and y are input variables (names for known data values), z is an output variable (a name for a data value to be computed), low is a membership function (fuzzy subset) defined on x, high is a membership function defined on y, and medium is a membership function defined on z.

The antecedent (the rule's premise) describes to what degree the rule applies, while the conclusion (the rule's consequent) assigns a membership function to each of one or more output variables. Most tools for working with fuzzy expert systems allow more than one conclusion per rule. The set of rules in a fuzzy expert system is known as the rulebase or knowledge base.

The general inference process proceeds in three (or four) steps.

1. Under FUZZIFICATION, the membership functions defined on the input variables are applied to their actual values, to determine the degree of truth for each rule premise.

2. Under INFERENCE, the truth value for the premise of each rule is computed, and applied to the conclusion part of each rule. This results in one fuzzy subset to be assigned to each output variable for each rule. Usually only MIN or PRODUCT are used as inference rules. In MIN inferencing, the output membership function is clipped off at a height corresponding to the rule premise's computed degree of truth (fuzzy logic AND). In PRODUCT inferencing, the output membership function is scaled by the rule premise's computed degree of truth.

3. Under COMPOSITION, all of the fuzzy subsets assigned to each output variable are combined together to form a single fuzzy subset for each output variable. Again, usually MAX or SUM are used. In MAX composition, the combined output fuzzy subset is constructed by taking the pointwise maximum over all of the fuzzy subsets assigned to variable by the inference rule (fuzzy logic OR). In SUM composition, the combined output fuzzy subset is constructed by taking the pointwise sum over all of the fuzzy subsets assigned to the output variable by the inference rule.

4. Finally is the DEFUZZIFICATION, which is used to convert the fuzzy output set to a crisp value. Two of the more common techniques are the CENTROID and MAXIMUM methods. In the CENTROID method, the crisp value of the output variable is computed by finding the variable value of the center of gravity of the aggregated membership functions for the fuzzy values of the output. In the MAXIMUM method, crisp value of the output value is selected from the fuzzy word of the highest membership function.

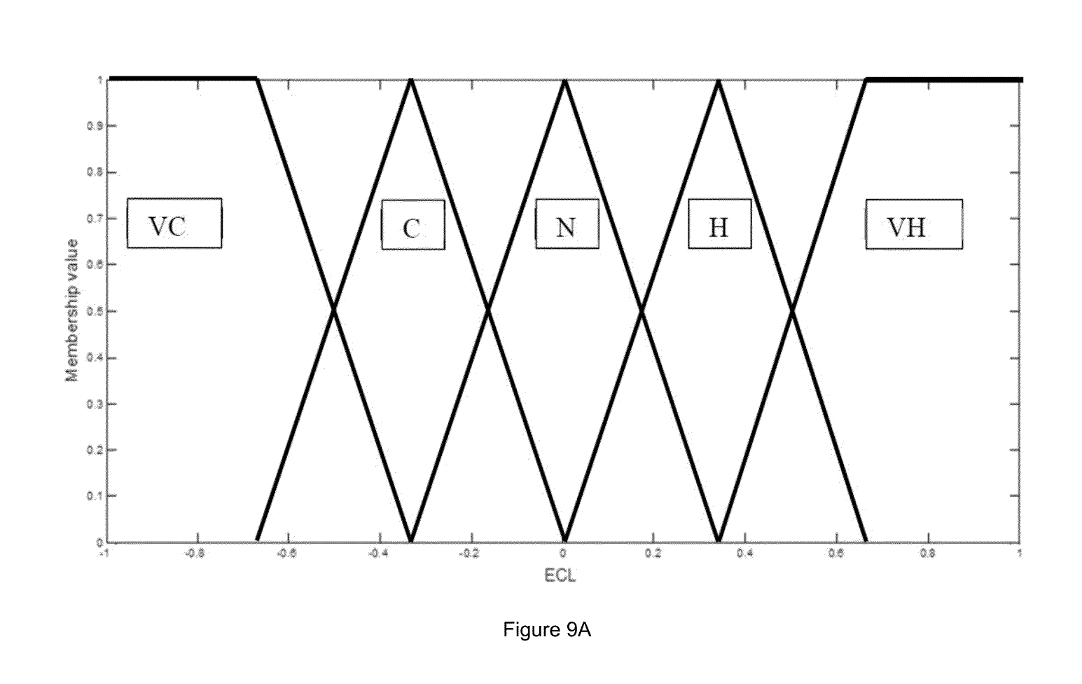

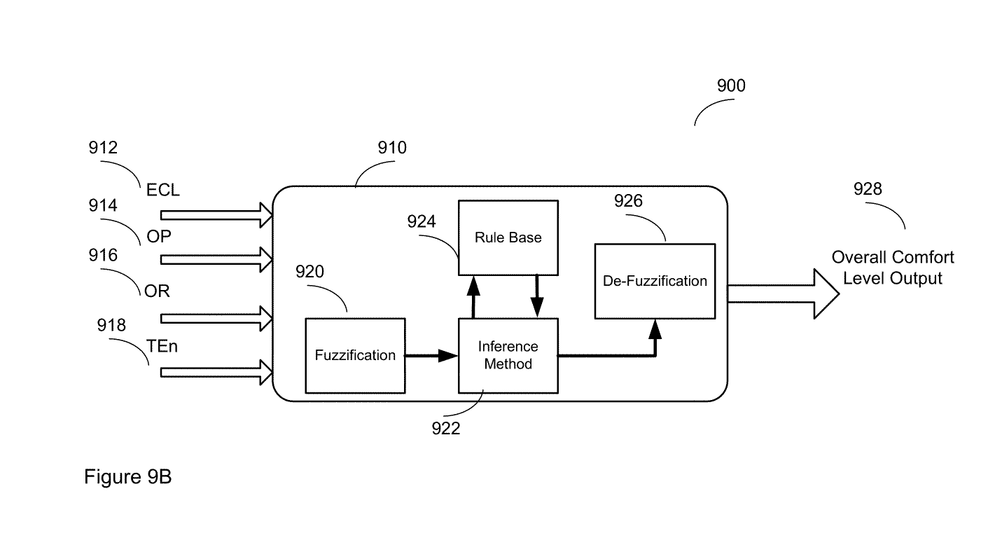

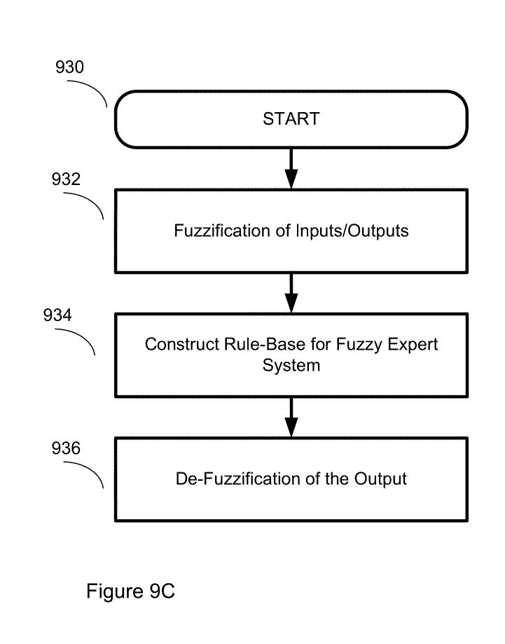

Crisp value represents the continuous value of a parameter. For example, ECL can have any value between (−1 to +1; which are referred to as the universe of discourse) as a continuous value. The range is then transformed into fuzzy words like Very cold (VC), cold (C), normal (N), hot (H) and very hot (VH) as shown in FIG. 9A. The membership value provides information as to how much fuzzy word belongs to a certain value of temperature. As such, the crisp value of ECL can be transformed into fuzzy words and their membership values. For example, if ECL is −0.6, then the conversion set will be {VC/0.8, C/0.2, N/0, H/0, VH/0} to illustrate the fuzzy word membership.

FIG. 9B is an illustration of a comfort level expert system according to an exemplary embodiment. Comfort level expert system comprises of fuzzy expert system such as Mamdani fuzzy expert system. Environmental comfort level (ECL) 912, overall pressure on the body (OP) 914, available oxygen ratio (OR) 916 and total energy expenditure from activity classification and logging circuit (TEn) 918 are the inputs to the fuzzy expert system 910. Fuzzification circuit 920 of the expert system 900 converts the crisp values of the inputs into proper fuzzy words defined on the universe of discourse. Fuzzification circuit 920 includes a plurality of membership function circuits (not shown). Included in the membership function circuits are an address decoder and a membership function RAM (not shown) having a plurality of memory locations. It should be noted that each of the plurality of membership function circuits is configured similarly.

The fuzzification circuit 920 performs fuzzy logic operations with a high degree of accuracy in a minimal amount of time. The high speed is attributed to an architecture which performs all fuzzy logic operations in a unary number system, rather than a more traditional binary number system. Because the fuzzification circuit 920 performs all fuzzification and rule evaluation steps (performed in rule base circuit 924) in unary, the only time required to perform each of these steps is equal to only a time required for the signal to propagate through the logic gates forming fuzzification circuit 920 and rule base circuit 924 of fuzzy expert system 910. No circuitry requiring clocks or timing is involved during execution of the fuzzification and rule evaluation steps and therefore, fuzzy expert system 910 is not limited by the speed at which it may be clocked. Rather fuzzy expert system 910 is limited only by the intrinsic delay of the logic circuitry therein.

Fuzzification is a first step in a fuzzy inference operation and is performed by fuzzification circuit 920. During the fuzzification step, fuzzification circuit 920 converts an input value into N fuzzy membership values, where N is a number of membership functions defined in an input space. The N fuzzy membership values are then used during a fuzzy rule evaluation step in the fuzzy inference operation. In the present embodiment of the invention, each membership function is represented by 32 memory locations. Therefore, when there are 256 possible inputs, 32 of the inputs access a particular membership function. An address decoder, such as 16, of the membership function RAM, such as 17, determines which block of 32 inputs accesses each membership function stored in the membership function RAM. Membership values for inputs which do not address this block of 32 memory locations will default to a value of zero.

Rule-base circuit 924 contains the rules for the calculation of the overall comfort level based on these inputs. Inference method circuit 922 decides the rules which are fired according to the input values. The output is then passed through a de-fuzzification circuit 926 and the consequence (output) of the rules which are fired by a particular set of inputs are aggregated to produce a crisp value of the overall comfort level 928.

In one exemplary embodiment a universe of discourse is defined for each input (912,914,916,918). For example, for ECL, the universal discourse is (−1) to (+1), for OR the universal discourse is from (0) to (+1), etc. Then appropriate fuzzy words for each input are decided and the range and shape of the membership function for each fuzzy word is decided as also described in FIG. 9A. This will complete the process of Fuzzification (920). For any set of inputs coming from outside of 910, Fuzzification will produce the membership value for each input to the fuzzy word set for this particular input.

The rule-base contains all the rules related to the fuzzy words of all the inputs done in Fuzzification. For example, the rule-base contains the following form:
Rule 1: IF ECL is VC AND OR is LOW then OCL is LOW
Rule 2: If ECL is H AND OR is LOW AND Ten is HIGH then OCL is HIGH
. . . .

In one example, if input values are −0.8, 200, 0.8, 100 for ECL, OP, OR, and TEn respectively, ECL is converted as VC/0.7, C/0.2, N/0, H/0 and VH/0; similarly OP is converted as High/0, Medium/0, Low/0.3, V-low/0.7 and so on.

Inference method 922 finds all the rules including all the fuzzy words having membership value greater than 0 and calculate the membership values for the output. De-fuzzification 926 applies the aggregation method to find out values of overall comfort level.

FIG. 9C illustrates a sample Fuzzification/defuzzification algorithm according to an exemplary embodiment. The Fuzzy expert system is constructed in the following algorithm,
Step 930: system is initiated and beings to receive the outputs of the sensors of the system. Outputs include ECL 912, OP 914, OR 916 and Ten 918.
Step 932: Fuzzification of the Inputs/Output: In this step, range of each input will be defined and appropriate fuzzy words will be defined for each input. Similarly output range and fuzzy words will be defined. One sample definition mechanism is further illustrated in FIG. 9B.
Step 934: Construct Rule-base of the fuzzy expert system. This rule-base includes rules that related the input and the output. An inference algorithm is used to predict the output (set of fuzzy words) based on a particular input and the rule-base.

Step 936: De-Fuzzification of the output: a crisp value of the output is constructed using aggregation method.

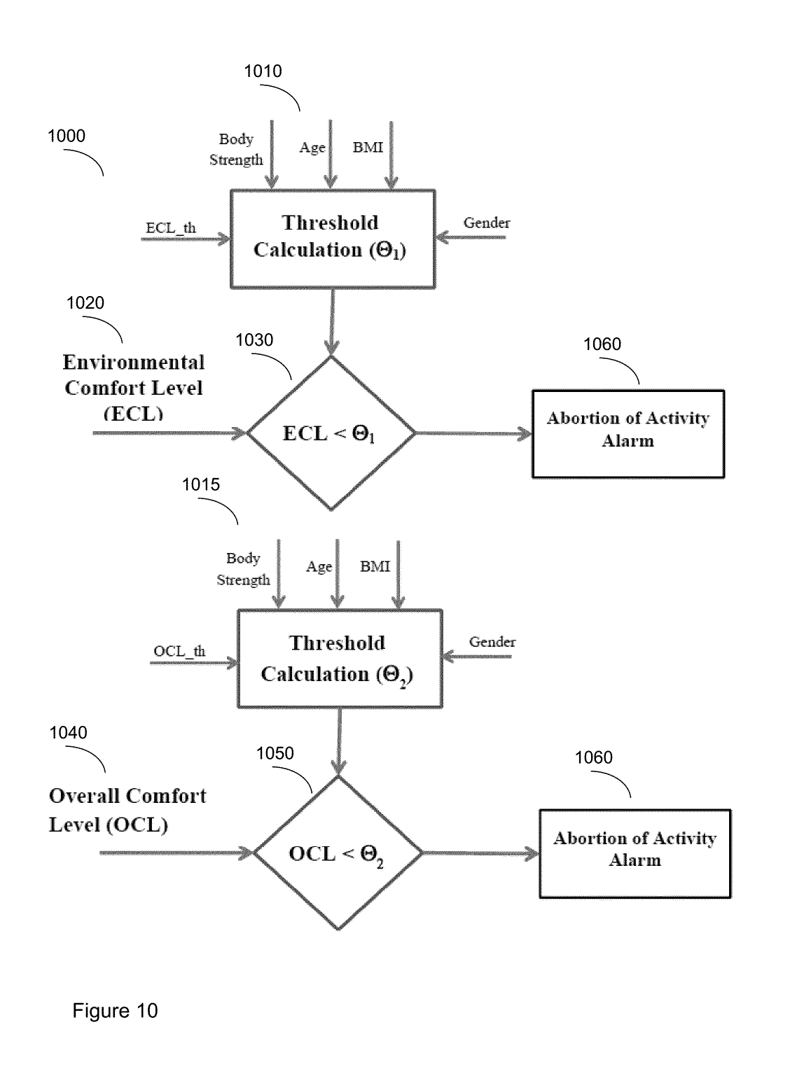

FIG. 10 is an illustration of a comfort level expert system with activity abortion recommendation according to an exemplary embodiment. As previously illustrated in FIG. 9, the overall comfort level output 928 is generated as a factor of several inputs, including an environmental comfort level, overall pressure on the body, available oxygen ratio and overall energy expenditure. Upon generation of an overall comfort level (OCL), the OCL and three other factors, including the environmental comfort level, oxygen ratio level and overall pressure on the body level are measured against respective thresholds, such that if factors violate their corresponding threshold value, then an alarm is generated to the user to abort the activity according to the situation.

For example, comfort level expert system 1000 may receive user inputs 1010. User inputs 1010 can be a variety of user inputs, including, for example, body strength, age, gender, BMI and a base threshold value such as a base threshold value for ECL, OCL, OR, and OP. For example, a base threshold value for OR is input, such as 0.5 to establish a minimum threshold, below which, the environment becomes dangerous and life threatening for persons in that environment. As such, when the minimum threshold is met, the activity abort alarm is automatically activated to encourage the individual to abort activity and seek safer environments. In another example, the individual may be part of a crowd and thus the crowd may be urged to seek safer environments by activating activity abort alarm that is deployed in the form of a public safety message, announcement, or sirens.

The base threshold is used in threshold calculation for each one of ECL, OCL, OP and OR. If ECL, OCL, OP or OR input levels violate the minimum threshold, then the activity abort alarm 1060 is automatically activated. If base threshold values are not respectively violated, then a threshold calculation is determined as a function of the user inputs 1010 or 1015. User inputs 1010 can then further affect the overall threshold calculation. For example, comfort level expert system 1000 can receive environmental comfort level (ECL) 1020 for evaluation. For the sake of illustration, assume that the base threshold ECL_th is calculated at an absolute value |0.70| (to remove the ± distinction for hot and cold discomfort). If ECL input value is greater than 0.70, activity abort alarm 1060 may be automatically activated. If ECL input value is below 0.70, for example 0.65, then the threshold calculation will modify the ECL associated threshold (Θ₁) based on the user inputs 1010. For example, if ECL=0.65, but age input is 80 and body strength reflects a “weak” measure, then ECL associated threshold (Θ₁) may be adjusted to (Θ₁)=0.60, thus resulting in activating the abort activity alarm 1060. Alternatively, if input age was 20, and body strength input reflects a “strong” or “very strong” measure, then ECL associated threshold (Θ₁) may be adjusted to a higher value.

As such, ECL 1020 is compared to the threshold Θ₁ at step 1030. If the ECL<Θᵢ then the activity abort alarm 1060 is activated and the individual is recommended to abort his/her activity and reach a safe place before the situation becomes even more dangerous.

In similar fashion, comfort level expert system 1000 can also receive inputs 1015 and calculate an OCL threshold (Θ₂) using the four user inputs 1010. Thereafter, OCL 1040 is compared to the threshold Θ₂ at step 1050. If OCL<Θ₂ then the abortion of activity alarm 1060 is activated and the individual is recommended to abort his/her activity and reach a safe place before the situation becomes even more dangerous.

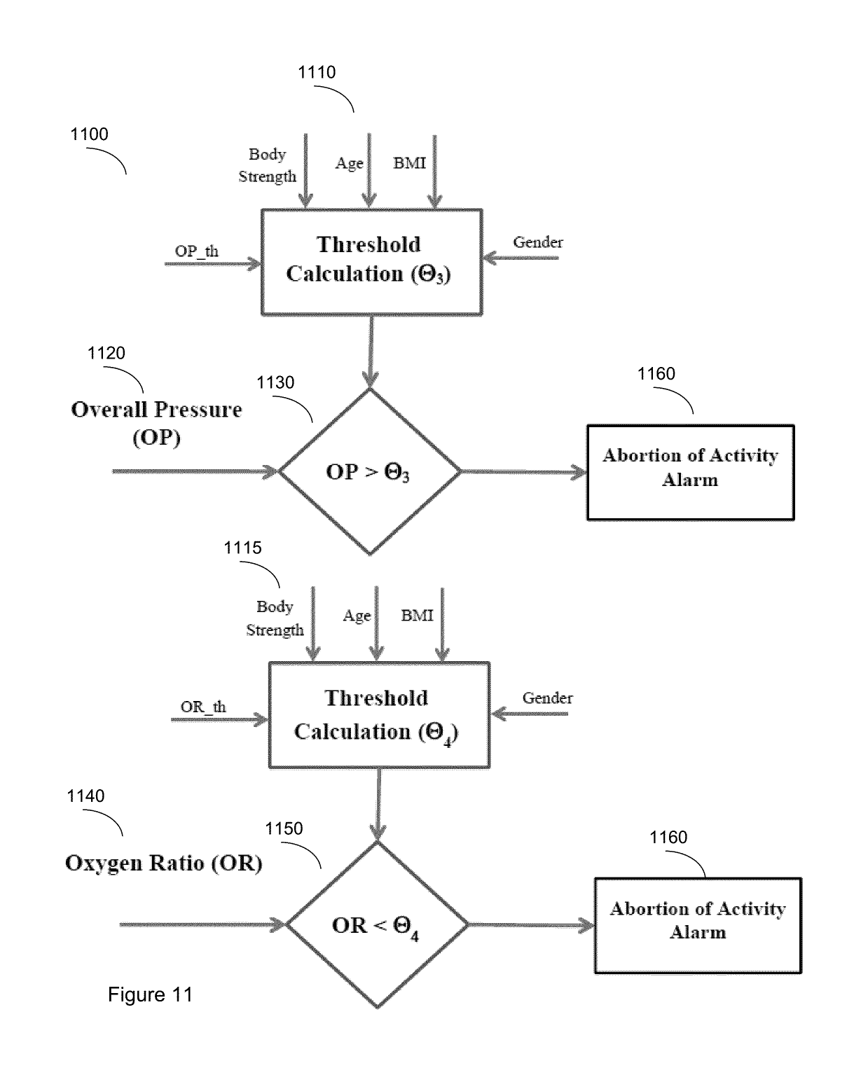

FIG. 11 is an illustration of a comfort level expert system with abortion of activity alarm generation according to another embodiment wherein two other factors are considered. For example, comfort level expert system 1000 can also receive inputs 1110 and calculate an overall pressure (OL) threshold (Θ₃) using the four user inputs 1110. Thereafter, OL 1120 is compared to the threshold Θ₃ at step 1130. If OL<Θ₃ then the activity abort alarm 1160 is activated and the individual is recommended to abort his/her activity and reach a safe place before the situation becomes even more dangerous. Additionally, comfort level expert system 1000 can also receive inputs 1115 and calculate an oxygen ratio (OR) threshold (Θ₄) using the four user inputs 1110. Thereafter, OR 1140 is compared to the threshold Θ₄ at step 1150. If OR<Θ₄ then activity abort alarm 1060 is activated and the individual is recommended to abort his/her activity and reach a safe place before the situation becomes even more dangerous. In some instances, threshold calculation may adjust threshold values above or below the base threshold value. For example, ECL threshold value (Θ_{f}) may be adjusted to be>|0.70| if the user inputs indicate a higher tolerance. In other instances, threshold calculations may not adjust the threshold values above or below the base threshold. For example, OR_th may be adjusted to 0.50 value to indicate a minimum acceptable oxygen level before serious health risks arise. Irrespective of user inputs, OR threshold (Θ₄) may not be adjusted below OR_th.

An example algorithm for abortion of activity alarm generation is illustrated in Table II below.

TABLE II
Algorithm of Activity Alarm generation
Algorithm: Abortion of Activity Alarm generation

Step 1: Calculation of Threshold values

Θ₁ = g₁ (Age, BMI, BS, ECL_th)
If gender = “Male” then Θ₁ = Θ₁ else Θ₁ = ζ₁ Θ₁
Θ₂ = g₂ (Age, BMI, BS, OCL_th)
If gender = “Male” then Θ₂ = Θ₂ else Θ₂ = ζ₂ Θ₂
Θ₃ = g₃ (Age, BMI, BS, OP_th)
If gender = “Male” then Θ₃ = Θ₃ else Θ₃ = ζ₃ Θ₃
Θ₄ = g₄ (Age, BMI, BS, OR_th)
If gender = “Male” then Θ₄ = Θ₄ else Θ₄ = ζ₄ Θ₄

Step 2: Alarm generation

If ECL< Θ₁ ∪ OCL < Θ₂ ∪ OP < Θ₃ ∪ OR < Θ₄ Then
Generate Alarm

Table II reflects the activity abort alarm generation algorithm in greater details. For example, in an initial step, threshold values are calculated for each of the four parameter inputs, including ECL threshold Θ₁, OCL threshold Θ₂, OP threshold Θ₃ and OR threshold Θ₄. The standard for each threshold calculation is a male standard, taking into account age, BMI, and body strength (BS). If the gender input by the individual is a female gender, then the threshold calculation is offset by a factor ζ to accommodate for physical attributes associated with the different genders.

An alarm is generated only if any of ECL, OCL, and OR are below their respective threshold values or OP is above its threshold value. As illustrated in step 2, a union of the factors is presented indicating that if any of the four conditions occurs, the alarm will be generated. Again, a generated alarm can be on the individual himself, on the application monitoring the individual, a siren alarm indicating crowd dispersion, or any combination of the above.

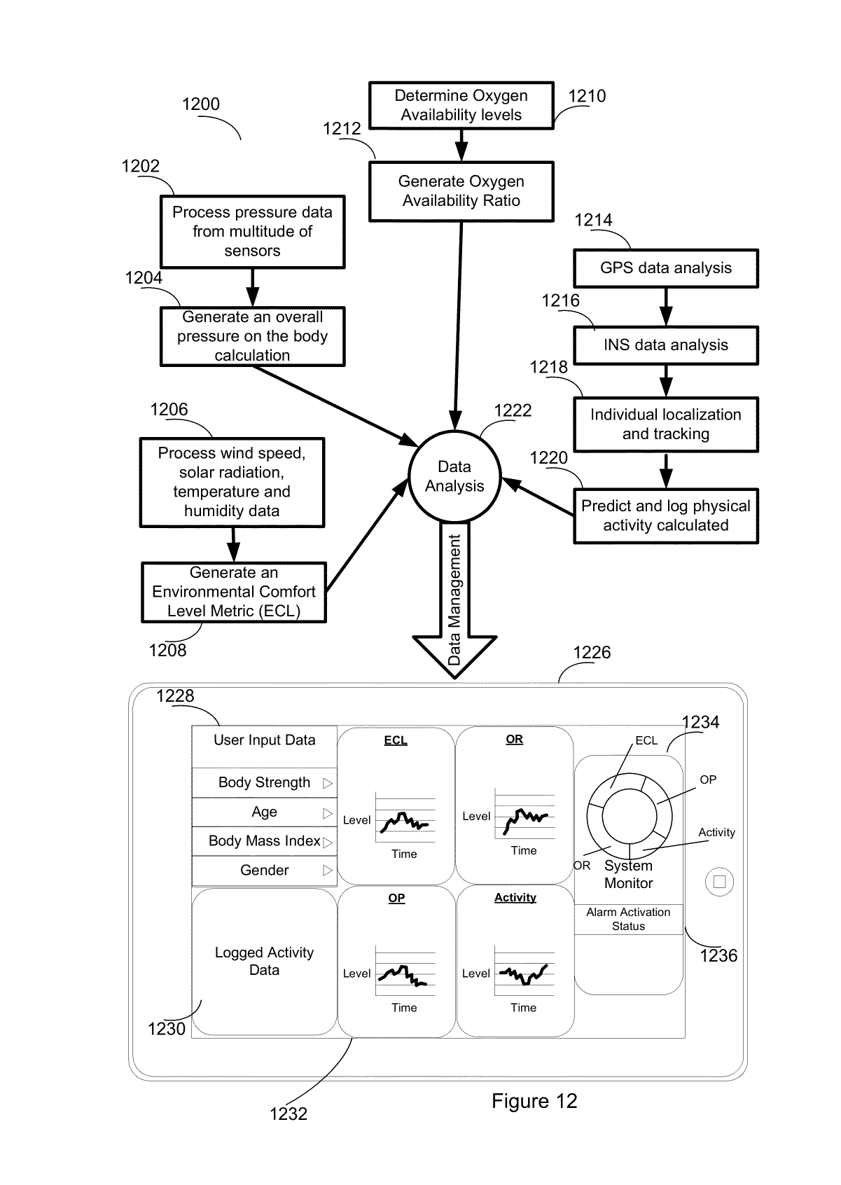

FIG. 12 is an illustration of an expert system management portal 1200 according to one embodiment. Expert system management portal 1200 includes processing pressure data from multitude of sensors 1202 and generating an overall pressure on the body (OP) calculation 1204. The calculation is then transmitted for data analysis 1222. Data analysis may be performed using any number of processors including, for example, data processing circuit 332 and data logger/expert system 104. Expert system management portal 1200 further includes processing wind speed, solar radiation, temperature and humidity 1206 to generate and environmental comfort level (ECL) 1208 that is passed on for further data analysis 1222. In addition, expert system management portal 1200 further includes determining oxygen availability levels 1210, using oxygen level sensor 326 for example, and generating oxygen availability ratio 1212 which is the passed on for data analysis 1222. Furthermore, GPS data analysis is performed 1214, INS data is analyzed 1216, individual localization and tracking 1218 is also performed to track the individual wearing the sensors and map out the individual's experiences, and predict and log calculated physical activity 1220 of the individual before sending for data analysis 1222.

Data analysis and management can be manipulated by a user interface device such as device 1226. User interface device 1226 may include data processing and data transmission and reception capabilities described in FIG. 3. User interface device 1226 may be anytype of mobile device, including, for example, an iPad, iPhone, or any other tablet computer or smart phone device. User interface device 1226 may include a user input interface 1228 adapted to include at least four user inputs, such as body strength, age, body mass index and gender of the user. User interface device 1226 further includes a logged activity data repository 1230 accessible to a user. Logged activity data repository 1230 may include a log of all activities recorded within a given period of time. The logged activity data can be useful in determining trends of individual travel, problematic geographic locations or travel times that produce heightened parameters or increased likelihood of alarm activations.

Status window 1232 illustrates level fluctuations over time of the four metrics used by the expert level system in determining an overall comfort level. For example, status window 1232 illustrates fluctuations of ECL, OP, OR and energy expenditure or activity levels over time. Threshold levels window 1234 may further illustrate a pie chart of different activities vs threshold analysis in an advanced way to monitor the system. Alarm activation status window 1236 enables the user to monitor the status of the alarm as well as track previously generated alarm activations for a given period of time. Alarm activation log may help determine crowd behavior and help a user deploy crowd control measures. For example, if it is determined that at a given time or place, or activity, or temperature, etc., an alarm is frequently triggered, it might be beneficial for the user to generate alternative routes for manage crowd traffic. Alternatively, if, during an event, such as a concert, a demonstration, a sports game, etc., an alarm is activated, sirens may further be deployed to effectuate disbursement. In other embodiments, location of the alarm triggering event may be used to determine if specific pockets within a crowd are generating above threshold pressure points, or below threshold oxygen levels, environmental comfort levels, or overall comfort levels. Such data can be important in not disrupting a whole crowd, but rather deploying personnel or measures to alleviate targeted pressure points.

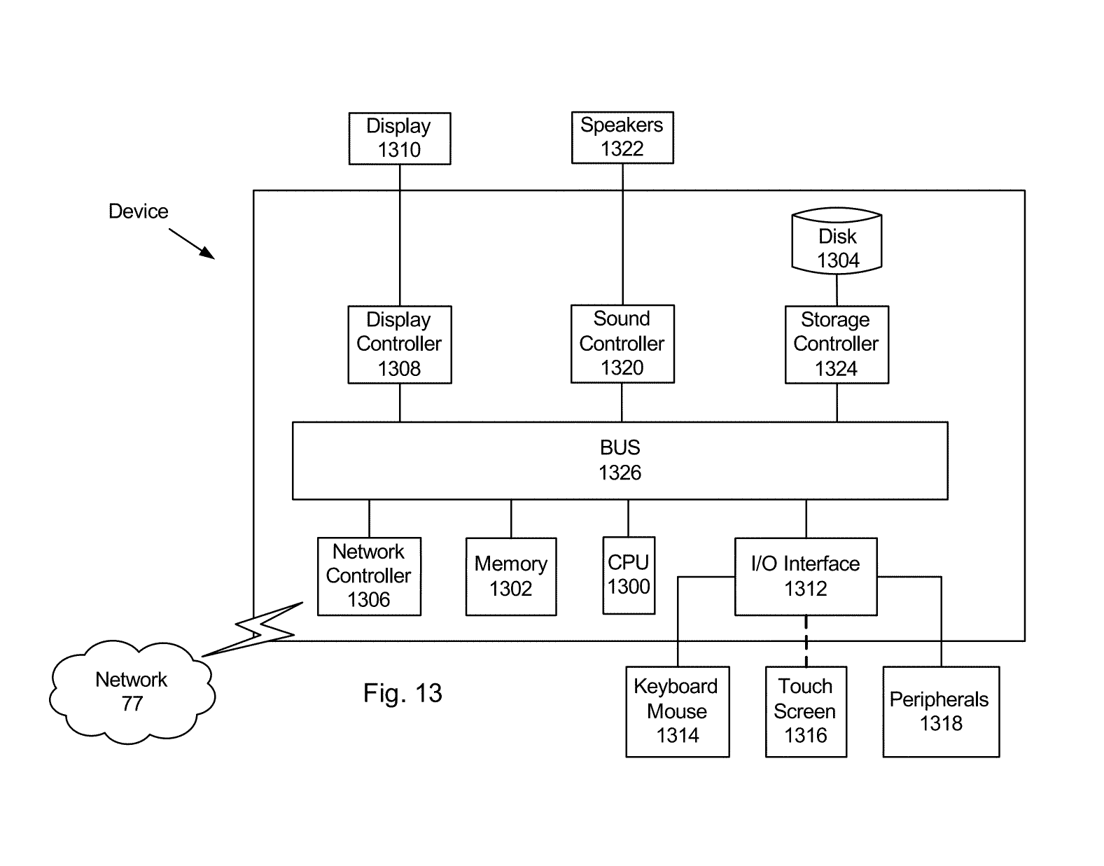

FIG. 13 an illustration of a hardware description of a device according to embodiments illustrated in FIGS. 1-12.

Next, a hardware description of a device according to exemplary embodiments illustrated in FIGS. 1-12 is described with reference to FIG. 13. In FIG. 13, the device includes a CPU 1300 which performs the processes described above. The process data and instructions may be stored in memory 1302. These processes and instructions may also be stored on a storage medium disk 1304 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device communicates, such as a server or computer.

Further, the present advancements may be provided as a utility application, background daemon, or component of an operating system, or combination thereof, executing in conjunction with CPU 1300 and an operating system such as Microsoft Windows 7, UNIX, Solaris, LINUX, Apple MAC-OS and other systems known to those skilled in the art.

CPU 1300 may be a Xenon or Core processor from Intel of America or an Opteron processor from AMD of America, or may be other processor types that would be recognized by one of ordinary skill in the art. Alternatively, the CPU 1300 may be implemented on an FPGA, ASIC, PLD or using discrete logic circuits, as one of ordinary skill in the art would recognize. Further, CPU 1300 may be implemented as multiple processors cooperatively working in parallel to perform the instructions of the inventive processes described above.

The device in FIG. 13 also includes a network controller 1306, such as an Intel Ethernet PRO network interface card from Intel Corporation of America, for interfacing with network 77. As can be appreciated, the network 77 can be a public network, such as the Internet, or a private network such as an LAN or WAN network, or any combination thereof and can also include PSTN or ISDN sub-networks. The network 77 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The device further includes a display controller 1308, such as a NVIDIA GeForce GTX or Quadro graphics adaptor from NVIDIA Corporation of America for interfacing with display 1310, such as a Hewlett Packard HPL2445w LCD monitor. A general purpose I/O interface 1312 interfaces with a keyboard and/or mouse 1314 as well as a touch screen panel 1316 on or separate from display 1310. General purpose I/O interface also connects to a variety of peripherals 1318 including printers and scanners, such as an OfficeJet or DeskJet from Hewlett Packard.

A sound controller 1320 is also provided in the device, such as Sound Blaster X-Fi Titanium from Creative, to interface with speakers/microphone 1322 thereby providing sounds and/or music.

The general purpose storage controller 1324 connects the storage medium disk 1304 with communication bus 1326, which may be an ISA, EISA, VESA, PCI, or similar, for interconnecting all of the components of the device. A description of the general features and functionality of the display 1310, keyboard and/or mouse 1314, as well as the display controller 1308, storage controller 1324, network controller 1306, sound controller 1320, and general purpose I/O interface 1312 is omitted herein for brevity as these features are known.

Thus, the foregoing discussion discloses and describes exemplary embodiments of the present disclosure for clarity. As will be understood by those skilled in the art, the present invention may be embodied in other specific forms without departing from the spirit or essential characteristics thereof and aspects of the exemplary embodiments described herein may be combined differently to form additional embodiments or omitted. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A comfort level monitoring system comprising:
a wearable item configured to house a plurality of sensors;
at least one physical pressure measuring sensor configured to detect an amount of external physical pressure exerted on a person wearing the wearable item;
a first set of sensors configured to detect environmental data associated with an environmental comfort level of the person wearing the wearable item;
a second set of sensors configured to detect total energy expenditure of the person;
a user interface configured to receive personal physical data associated with the person;
a fuzzy logic circuit configured to apply a fuzzy logic algorithm to calculate, in response to received inputs from the at least one physical pressure measuring sensor, the first set of sensors and the second set of sensors, an overall comfort level associated with the person; and
an activity alarm configured to be triggered when the overall comfort level falls below a predetermined threshold.

2. The comfort level monitoring system of claim 1, wherein the at least one physical pressure measuring sensors further comprises at least four physical pressure measuring sensors placed within the wearable item to maximize measurement of external physical pressure.

3. The comfort level monitoring system of claim 2, wherein a different value weighting is assigned to each one of the at least four physical pressure measuring sensors depending on a location of the at least four physical pressure measuring sensor within the attire.

4. The comfort level monitoring system of claim 1, wherein the first set of sensors includes at least one of a temperature sensor, a humidity level sensor, a wind speed sensor, and a solar radiation sensor.

5. The comfort level monitoring system of claim 4, wherein the environmental comfort level is generated by non-linear aggregating circuitry configured to create an n dimensional space, for n input parameters.

6. The comfort level monitoring system of claim 5, wherein when n=4, the input parameters include wind speed information, solar radiation information, temperature information and humidity information.

7. The comfort level monitoring system of claim 1, wherein the second set of sensors further includes at least one global positioning sensor (GPS) and at least one inertial navigation sensor (INS).

8. The comfort level monitoring system of claim 7, wherein the GPS and INS sensor data is used to predict an activity performed by the person and the total energy expenditure calculation is dependent on the predicted activity performed by the person.

9. The comfort level monitoring system of claim 8, wherein the predicted activity includes walking, running, standing, and sitting.

10. The comfort level monitoring system of claim 1, wherein the personal physical data associated with the person includes body strength, age, body mass index (BMI) and gender.

11. The comfort level monitoring system of claim 10, wherein a different value weighting is assigned to each one of the genders such that the generated overall comfort level is dependent on the weighting of the different genders.

12. The comfort level monitoring system of claim 1, wherein the activity abort alarm is further configured to instruct at least one individual to abort the physical activity currently in pursuit.

13. The comfort level monitoring system of claim 1, the activity abort alarm further generates a crowd dispersion outline configured to ease specific congestion location within a venue.

14. A comfort level monitoring method comprising:
housing a plurality of sensors in a wearable item;
circuitry configured to
detect an amount of external physical pressure exerted on a person wearing the wearable item,
measure environmental data associated with an environmental comfort level of the person wearing the wearable item,
calculate oxygen ratios associated with the environment surrounding the person,
measure total energy expenditure of the person,
receive and process personal physical data associated with the person,
apply a fuzzy logic algorithm to calculate, in response to received inputs, an overall comfort level associated with the person, and
trigger an activity abort alarm when the overall comfort level falls below a predetermined threshold.

15. The comfort level monitoring method of claim 14, wherein the circuitry further comprises at least four physical pressure measuring sensors placed within the wearable item to maximize measurement of external physical pressure.

16. The comfort level monitoring method of claim 15, wherein a different value weighting is assigned to each one of the at least four physical pressure measuring sensors depending on a location of the at least four physical pressure measuring sensor within the attire.

17. The comfort level monitoring method of claim 14, wherein the circuitry further comprises at least one of a temperature sensor, a humidity level sensor, a wind speed sensor, and a solar radiation sensor.

18. A non-transitory computer-readable storage medium having computer readable program codes embodied in the computer readable storage medium that, when executed cause a computer to execute a display control method for controlling a display control device comprising:
housing a plurality of sensors in a wearable item;
detecting an amount of external physical pressure exerted on a person wearing the wearable item;
measuring environmental data associated with an environmental comfort level of the person wearing the wearable item;
calculating oxygen ratios associated with the environment surrounding the person;
measuring total energy expenditure of the person;
receiving and process personal physical data associated with the person;
applying a fuzzy logic algorithm to calculate, in response to received inputs, an overall comfort level associated with the person; and
triggering an activity abort alarm when the overall comfort level falls below a predetermined threshold.

19. The non-transitory computer-readable storage medium of claim 18, wherein detecting an amount of external physical pressure further includes generating inputs from at least four physical pressure measuring sensors placed within the wearable item to maximize measurement of external physical pressure.

20. The non-transitory computer-readable storage medium of claim 19, wherein a different value weighting is assigned to each one of the at least four physical pressure measuring sensors depending on a location of the at least four physical pressure measuring sensor within the attire.
